(19) United States
(12) Patent Application Publication
(10) Pub. No.: US 2022/0351822 A1
(43) Pub. Date: Nov. 3, 2022

(54) TECHNIQUES FOR EXECUTING AND MODIFYING TRANSIENT CARE PLANS VIA AN INPUT/OUTPUT DEVICE

(71) Applicant: Intuition Robotics, Ltd, Ramat-Gan (IL)

(72) Inventors: Shay ZWEIG, Harel (IL); Alex KEAGEL, Tel Aviv (IL); Chen SORIAS, Kibbutz Zikim (IL); Dor SKULER, Oranit (IL)

(73) Assignee: Intuition Robotics, Ltd., Ramat-Gan (IL)

(21) Appl. No.: 17/661,787

(22) Filed: May 3, 2022

Related U.S. Application Data
(60) Provisional application No. 63/183,343, filed on May 3, 2021.

(51) Int. Cl. G16H 20/30

(52) U.S. Cl. CPC G16H 20/30

(57) ABSTRACT

Techniques for executing transient care plans are provided. A transient care plan to be executed via the I/O device is created based on analysis of an initial care plan of a user and a user dataset captured by an I/O device. The initial care plan is a health regimen uniquely determined for the user indicating medical instructions defined with respect to a first frequency. The transient care plan defines a second frequency at which instructions to perform the user activity are to be projected via the I/O device. The transient care plan is executed by projecting the instructions based on the second frequency. The transient care plan is modified based on a change in status of the user to define a third frequency. The modified transient care plan is executed by projecting the instructions to perform the user activity via the I/O device based on the third frequency.

### CROSS-REFERENCE TO RELATED APPLICATIONS

This application claims the benefit of U.S. Provisional Application No. 63/183,343 filed on May 3, 2021, the contents of which are hereby incorporated by reference.

### TECHNICAL FIELD

The disclosure generally relates to digital assistants operated via an input/output (I/O) device, and more specifically to techniques for executing and modifying transient care plans for a user of I/O devices.

### BACKGROUND

As manufacturers improve the functionality of devices such as vehicles, computers, mobile phones, appliances, and the like, through the addition of digital features, manufacturers and end-users may desire enhanced device functionalities. The manufacturers, as well as the relevant end-users, may desire digital features which improve user experiences, interactions, and features which provide for greater connectivity. Certain manufacturers may include device-specific features, such as setup wizards and virtual assistants, to improve device utility and functionality. Further, certain software packages may be added to devices, either at the point of manufacture, or by a user after purchase, to improve device functionality. Such software packages may provide functionalities including, as examples, a computer system's voice control, facial recognition, biometric authentication, and the like.

While the features and functionalities described hereinabove provide for certain enhancements to a user's experience when interacting with a device, the same features and functionalities, as may be added to a device by a user or manufacturer, fail to include certain aspects which may allow for a further-enhanced user experience. First, certain currently-implemented digital assistants and other user experience features may fail to provide for adaptive adjustment of the operation of the assistant or feature. For example, a digital assistant configured to play music may be programmed to use a specific type of music streaming services, thereby limiting the user experience. In addition, certain currently-implemented digital assistants and other user experience features may fail to provide for adjustments of the assistant or feature operation based on context or environmental data. As an example, a digital assistant may be configured to present reminders to take vitamins at a certain time. However, such reminder may be inappropriate and disturbing when the user is surrounded by guests or at certain hours of the day.

Furthermore, certain currently-implemented digital assistants and other user experience features may fail to provide for adjustments of the assistant or feature operation with respect to individual users and their continuously changing needs. For example, a digital assistant may be configured to present a reminder to a user and present the same reminder for one year until manually modified by the user. It should be noted that user's needs change dynamically and continuously depending on, for example, but is not limited to, schedules, habits, preferences, goals, physical states, and the like.

Particularly, such adaptive adjustments are important and useful for assisting users in health-related operations that are closely related to the well-being of a user. In this case, adaptive adjustments are desired not only for changes in individual user needs, but also for each of the multiple users that can have vastly different health-related conditions. To this end, an adaptive adjustment of operation is desired for effectively and consistently assisting multiple users of the digital assistance. In the absence of such adaptiveness between each of the multiple users and their needs, the users may not benefit from policies or actions performed or suggested by the digital assistants, and eventually abandon the usage of such devices.

It would therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for executing transient care plans for a user via an input/output (I/O) device. The method comprises: analyzing an initial care plan of a user and at least one user dataset captured by an input/output (I/O) device, wherein the initial care plan of the user is a health regimen uniquely determined for the user indicating at least one medical instruction defined at least with respect to a first frequency of a user activity; creating a transient care plan to be executed via the I/O device based on the analysis, wherein the transient care plan defines a second frequency at which a plurality of instructions to perform the user activity is to be projected via the I/O device, wherein the second frequency is determined based on the first frequency and the at least one user dataset captured by the I/O device; executing the transient care plan by projecting the plurality of instructions to perform the user activity via the I/O device based on the second frequency; modifying the transient care plan based on data indicating a change in status of the user, wherein the modified transient care plan defines a third frequency at which a plurality of instructions to perform the user activity is to be projected via the I/O device; and executing the modified transient care plan by projecting the plurality of instructions to perform the user activity via the I/O device based on the third frequency.

Certain embodiments disclosed herein also include a non-transitory computer readable medium having stored thereon causing a processing circuitry to execute a process, the process comprising: analyzing an initial care plan of a user and at least one user dataset captured by an input/output (I/O) device, wherein the initial care plan of the user is a health regimen uniquely determined for the user indicating at least one medical instruction defined at least with respect to a first frequency of a user activity; creating a transient care plan to be executed via the I/O device based on the analysis, wherein the transient care plan defines a second frequency at which a plurality of instructions to perform the user activity is to be projected via the I/O device, wherein the second frequency is determined based on the first frequency and the at least one user dataset captured by the I/O device; executing the transient care plan by projecting the plurality of instructions to perform the user activity via the I/O device based on the second frequency; modifying the transient care plan based on data indicating a change in status of the user, wherein the modified transient care plan defines a third frequency at which a plurality of instructions to perform the user activity is to be projected via the I/O device; and executing the modified transient care plan by projecting the plurality of instructions to perform the user activity via the I/O device based on the third frequency.

Certain embodiments disclosed herein also include a system for executing transient care plans for a user via an input/output (I/O) device. The system comprises: a processing circuitry; and a memory, the memory containing instructions that, when executed by the processing circuitry, configure the system to: analyze an initial care plan of a user and at least one user dataset captured by an input/output (I/O) device, wherein the initial care plan of the user is a health regimen uniquely determined for the user indicating at least one medical instruction defined at least with respect to a first frequency of a user activity; create a transient care plan to be executed via the I/O device based on the analysis, wherein the transient care plan defines a second frequency at which a plurality of instructions to perform the user activity is to be projected via the I/O device, wherein the second frequency is determined based on the first frequency and the at least one user dataset captured by the I/O device; execute the transient care plan by projecting the plurality of instructions to perform the user activity via the I/O device based on the second frequency; modify the transient care plan based on data indicating a change in status of the user, wherein the modified transient care plan defines a third frequency at which a plurality of instructions to perform the user activity is to be projected via the I/O device; and execute the modified transient care plan by projecting the plurality of instructions to perform the user activity via the I/O device based on the third frequency.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a network diagram utilized to describe the various embodiments of the disclosure.

FIG. 2 is a block diagram of a controller, according to an embodiment.

FIG. 3 is a flowchart illustrating a method for executing and modifying a transient care plan for a user of a digital assistant according to an embodiment.

FIG. 4 is a flowchart illustrating a method for adjusting a transient care plan based on feedback data received from the user of a digital assistant with respect to the presented transient care plan according to an embodiment.

FIG. 5 is a flowchart illustrating a method for detecting a first gap between an initial care plan and a transient care plan and for generating a corresponding electronic report according to an embodiment.

FIG. 6 is a flowchart illustrating a method for detecting a second gap between a transient care plan and a user performance data according to an embodiment.

### DETAILED DESCRIPTION

The embodiments disclosed by the disclosure are only examples of the many possible advantageous uses and implementations of the innovative teachings presented herein. In general, statements made in the specification of the present application do not necessarily limit any of the various claimed disclosures. Moreover, some statements may apply to some inventive features but not to others. In general, unless otherwise indicated, singular elements may be in plural and vice versa with no loss of generality. In the drawings, like numerals refer to like parts through several views.

The disclosed embodiments improve I/O devices utilized to execute these plans. In particular, an initial care plan for the user requiring performance of a user activity at a first frequency is analyzed with respect to user data for the user in order to create a transient care plan at least requiring projection of certain instructions to perform user activities at a second frequency. In this regard, the transient care plan may initially be customized based on known user behavior or preferences to project at a frequency that differs from a frequency proposed by a medical provider. Over time, the transient care plan is modified based on changes in status of the user in order to produce a modified transient care plan that at least requires projection of certain instructions to perform user activities at a third frequency. The result is that outputs of the I/O device are adjusted over time, particularly but not necessarily only with respect to frequency, in order to optimize the effectiveness of these outputs.

Moreover, the disclosed embodiments provide an objective process for adjusting transient plans over time, particularly with respect to frequencies of projection for the plans or portions thereof. In this regard, it is noted that humans may make plans to be executed via I/O devices and attempt to execute the plans accordingly, but that these plans are not executed consistently because they are created and/or modified based on subjective opinions about what the plans “should” be and how they should accommodate changes in the users' activity and schedules. Even when an initial plan is created using objective criteria, modifications to the plan are often made based on subjective evaluations of progress or preferences. Consequently, any attempts to establish routines via I/O devices, which typically require consistency, either fail or are successful only in spite of the operation of the I/O devices.

The disclosed embodiments avoid this inconsistent modification and execution of plans by utilizing objective rules for creating and modifying plans based on inputs to an I/O device and other objective markers of user status such as predetermined milestones of progress rather than only based on subjective user intentions. The result is that plans are executed more by I/O devices in a manner that is consistent among different I/O devices.

Various disclosed embodiments further include techniques for determining current states of users based on user and environment data captured via the I/O device as well as techniques for using such current states of users in order to modify transient care plans. Thus, the disclosed embodiments allow for continually or repeatedly refining execution of the plans via the I/O device as the I/O device collects more data about the user and the environment in which the I/O device is deployed.

Additionally, the disclosed embodiments allow for effectuating treatment plans for patients requiring consistent performance of certain user activities through I/O devices. To this end, an activity regimen prescribed by a doctor may be analyzed in order to create the transient care plan and adjusted to maximize compliance with the original prescription. As the transient care plan is adjusted over time based on user progress and/or compliance with the transient care plan, the frequency at which the I/O device projects instructions to perform certain user activities may likewise be adjusted. This, in turn, tunes the frequency used by the I/O device in order to maximize user compliance with the activity regimen, thereby improving treatment of the user via performance of the activity regimen

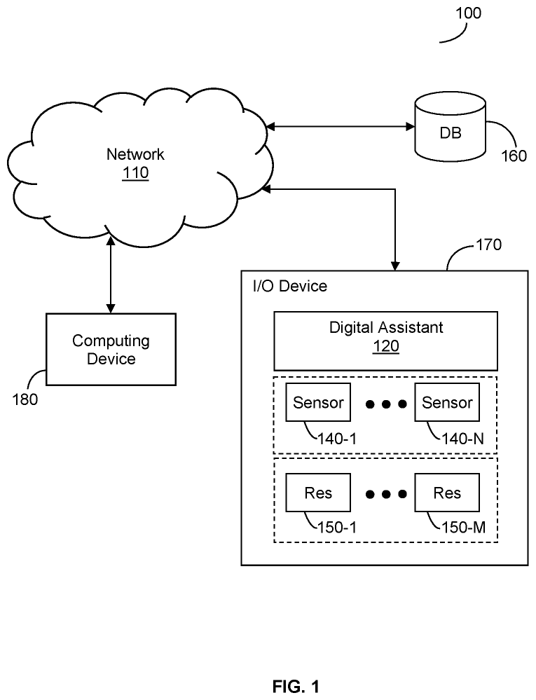

FIG. 1 is an example network diagram 100 utilized to describe the various disclosed embodiments. The network diagram 100 includes an input/output (I/O) device 170 operating a digital assistant 120. In some embodiments, the digital assistant 120 is further connected to a network 110 to allow some processing of a remote server (e.g., a cloud server). The network 110 may provide for communication between the elements shown in the network diagram 100. The network 110 may be, but is not limited to, a local area network (LAN), a wide area network (WAN), a metro area network (MAN), the Internet, a wireless, cellular, or wired network, and the like, and any combination thereof.

In an embodiment, the digital assistant 120 may be connected to, or implemented on, the I/O device 170. The I/O device 170 may be, for example and without limitation, a robot, a social robot, a service robot, a smart TV, a smartphone, a wearable device, a vehicle, a computer, a smart appliance, and the like.

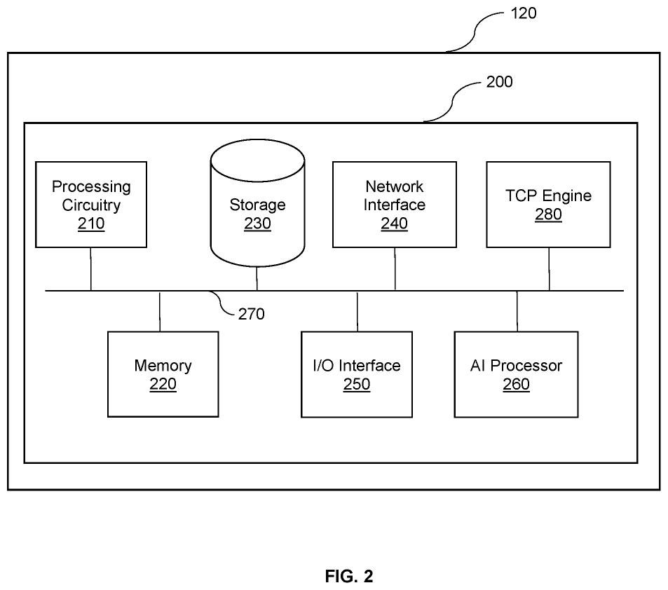

The digital assistant 120 may be realized in software, firmware, hardware, and any combination thereof. An example block diagram of a controller that may execute the processes of the digital assistant 120 is provided in FIG. 2. The digital assistant 120 is configured to process sensor data collected by one or more sensors, 140-1 to 140-N, where N is an integer equal to or greater than 1 (hereinafter referred to as “sensor” 140 or “sensors” 140 for simplicity) and one or more resources 150-1 to 150-M, where M is an integer equal to or greater than 1 (hereinafter referred to as “resource” 150 or “resources” 150 for simplicity). The resources 150 may include, for example, electro-mechanical elements, display units, speakers, and the like. In an embodiment, the resources 150 may include sensors 140 as well. The sensors 140 and the resources 150 are included in the I/O device 170.

The sensors 140 may include input devices, such as various sensors, detectors, microphones, touch sensors, movement detectors, cameras, and the like. Any of the sensors 140 may be, but are not necessarily, communicatively, or otherwise connected to the digital assistant 120 (such connection is not illustrated in FIG. 1 for the sake of simplicity and without limitation on the disclosed embodiments). The sensors 140 may be configured to sense signals received from a user interacting with the I/O device 170 or the digital assistant 120, signals received from the environment surrounding the user, and the like. In an embodiment, the sensors 140 may be implemented as virtual sensors that receive inputs from online services, for example, the weather forecast, a user's calendar, and the like.

In an embodiment, a database (DB) 160 may be utilized. The database 160 may be part of the I/O device 170 (e.g., within a storage device not shown), or may be separate from the I/O device 170 and connected thereto via the network 110. The database 160 may be utilized for storing, for example, health-related information of one or more users of the digital assistant, predefined health-related guidelines of the digital assistant, historical information of the user (e.g., patterns, preferences, and so on), and the like, as well as any combination thereof.

In an embodiment, a computing device 180 may be communicatively connected to the I/O device 170 via the network 110. The computing device 180 may be a personal computer (PC), a laptop, a server, and the like that is associated with an entity such as, but not limited to, a medical entity. A medical entity, or medical provider, may be for example, a medical team, a doctor, and the like. The computing device 180 may be configured to communicate with the digital assistant 120 through the network 110.

FIG. 2 is an example block diagram of a controller 200 acting as a hardware layer of a digital assistant 120, according to an embodiment. The controller 200 includes a processing circuitry 210 that is configured to receive data, analyze data, generate outputs, and the like, as further described hereinbelow. The processing circuitry 210 may be realized as one or more hardware logic components and circuits. For example, and without limitation, illustrative types of hardware logic components that can be used include field programmable gate arrays (FPGAs), application-specific integrated circuits (ASICs), application-specific standard products (ASSPs), system-on-a-chip systems (SOCs), general-purpose microprocessors, microcontrollers, digital signal processors (DSPs), and the like, or any other hardware logic components that can perform calculations or other manipulations of information.

The controller 200 further includes a memory 220. The memory 220 may contain therein instructions that, when executed by the processing circuitry 210, can cause the controller 200 to execute actions as further described hereinbelow. The memory 220 may further store therein information, for example but not limited to, data associated with one or more users, health-related information of one or more users, predetermined health parameters, values associated with the health parameters, historical information of the user (e.g., patterns, preferences, etc.), various feedback data (e.g., a user response, a user reaction, etc.), and the like.

The storage 230 may be magnetic storage, optical storage, and the like, and may be realized, for example, as a flash memory or other memory technology, or any other medium which can be used to store the desired information.

In an embodiment, the controller 200 includes a network interface 240 that is configured to connect to a network, e.g., the network 110 of FIG. 1. The network interface 240 may include, but is not limited to, a wired interface (e.g., an Ethernet port) or a wireless port (e.g., an 802.11 compliant Wi-Fi card), configured to connect to a network (not shown).

The controller 200 further includes an input/output (I/O) interface 250 configured to control the resources (e.g., 150, FIG. 1) which are connected to the digital assistant 120. In an embodiment, the I/O interface 250 is configured to receive one or more signals captured by the sensors (e.g., 140, FIG. 1) of the digital assistant (e.g., 120, FIG. 1) and to send such signals to the processing circuitry 210 for analysis. In an embodiment, the I/O interface 250 is configured to analyze the signals captured by the sensors 140, detectors, and the like. In a further embodiment, the I/O interface 250 is configured to send one or more commands to one or more of the resources 150 for executing one or more transient care plans (e.g., actions) of the digital assistant 120, as further discussed herein below. A care plan is a health regimen that is uniquely determined for a person such as the user of the digital assistant 120. An initial care plan is determined based on medical instructions to project certain instructions for a user to perform a user activity at a first frequency as proposed by a medical provider. As an example, a plan may include suggesting the user to timely take her or his medications, play a cognitive game, suggesting the user to go out for a walk, reminding the user to go to yoga class, and so on.

The transient care plan may be determined based on the unique care plan for further personalization for each of the users of the digital assistant 120. A transient care plan is a health regimen that is customized to the user based on, among other things, the user's behavior, patterns, habits, preferences, and the like, as further discussed herein below. In an embodiment, the transient care plan includes a plurality of instructions to perform user activities to be projected via an I/O device executing the digital assistant 120 according to a frequency. The transient care plan is generated based on the initial care plan and changes over time. In an embodiment, the transient care plan may include multiple subplans. It should be noted that the transient care can be realized as a single care plan that is updated to create multiple versions of the same overlying plan or multiple plans that are generated as entirely new plans which collectively serve as a transient care plan.

In some configurations, the controller 200 may further include an artificial intelligence (AI) processor 260. The AI processor 260 may be realized as one or more hardware logic components and circuits, including graphics processing units (GPUs), tensor processing units (TPUs), neural processing units, vision processing units (VPU), reconfigurable field-programmable gate arrays (FPGA), and the like. The AI processor 260 is configured to perform, for example, machine learning based on sensory inputs received from the I/O interface 250, where the I/O interface 250 receives input data, such as sensory inputs, from the sensors 140.

In an embodiment, the controller 200 may further include a transient care plan (TCP) engine 280. The TCP engine 280 is used for determining a care plan that is customized to the user based on inputs such as, the user's information, collected real-time data and current state of the user and the environment near the user, as further discussed herein. The TCP engine 280 is configured to receive and analyze several datasets (such as initial care plan, predefined health-related guidelines of the digital assistant 120, and dataset gathered with respect to the user) in order to determine and generate a transient care plan.

A transient care plan is a health regimen (e.g., a user activity) that is customized to the user based on, among other things, the user's behavior, patterns, habits, preferences, and the like as further discussed herein below. In an embodiment, the transient care plan includes a plurality of instructions on a user activity to be projected to a user via an I/O device 170 according to a second frequency. The second frequency of the transient care plan may be a customized frequency based on the initial care plan and the user datasets captured via the I/O device 170. The TCP engine 280 is further configured to update the transient care plan by analyzing datasets such as, but not limited to, a change in status of the user, user response to the executed transient care plan, and the like, and any combination thereof. The updated transient care plan may define a third frequency at which the plurality of instructions on the user activity is to be projected via an I/O device 170.

In an embodiment, the components of the controller 200 are connected via a bus 270.

In an embodiment, the controller 200 may receive from a computing device (e.g., the computing device 180, FIG. 1) that is associated with a medical provider, an initial care plan that is related to the user. As noted above, a care plan is a health regimen that is uniquely determined for a person such as the user of the digital assistant 120. The initial care plan may include medical instructions such as, take a certain medication at a specific timing, do physical activity three times a week, avoid red meat, and so on. In a further embodiment, the initial care plan may include a first frequency associated with at least one medical instruction indicating the number of times in which the at least one medical instruction should be performed by the user. In an embodiment, the initial care plan of the user may be sent from the computing device 180 through the network 110 to the digital assistant 120 using a web interface. According to a further embodiment, the initial care plan of the user may be fed into an electronic medical record (EMR) of the user and thus, the digital assistant 120 may be configured to collect the care plan (or updates related to the transient care plan) from the EMR of the user.

In an embodiment, the controller 200, when executing the digital assistant 120, may be configured to analyze the initial care plan with at least a dataset of the user and a set of predefined health-related guidelines of the digital assistant 120. A dataset of the user may include at least historical data of the user (such as, preferences, behavioral patterns, and so on). In an embodiment, the dataset may also include real-time data that is being collected in real-time with respect to the user and the user's environment in a predetermined proximity by the I/O device 170.

The dataset of the user may be collected through time using sensors (e.g., the sensors 140) that are communicatively connected to and controlled by the digital assistant 120. That is, the dataset may include sensory inputs that are analyzed using, for example, machine learning algorithm that is adapted to interpret the meaning of certain scenarios (e.g., user is alone, has company, asleep, angry, sad, happy, watching TV, and the like). By collecting and analyzing sensory inputs that may be stored in a database (e.g., the database 160, FIG. 1), user preferences, habits, behavioral patterns, and the like may be determined. Thus, the dataset may be indicative of the user's preference, behavioral patterns, and the like.

The set of predefined health-related guidelines of the digital assistant 120 may refer to predetermined rules. Such health-related rules or guidelines may indicate for example that: the user should drink 6-8 glasses of water per day, the user should sleep at least 7 hours at night, the user should not be inactive for more than four hours in a row during daytime, and so on. The analysis of the initial care plan with the dataset of the user and the set of predefined health-related guidelines may be achieved by applying at least one algorithm, such as a machine learning algorithm, that is adapted to determine a transient care plan. A transient care plan may refer to a health regimen that is customized to the user based on, among other things, the user's behavior, patterns, habits, preferences, and the like. The transient care plan is further influenced from the initial care plan as determined by the medical provider and from the predefined health-related guidelines of the digital assistant 120. It should be noted that the transient care plan should be as similar as possible, and preferably identical, to the care plan of the medical provider (i.e., the initial care plan). To that end, the desired similarity between the transient care plan and the initial care plan may be predetermined as a predefined goal of the digital assistant 120. Components of a care plan (either the initial care plan or the transient care plan) may include, for example and without limitation, type of physical activity the user is required to perform, the frequency in which the physical activity should be performed, the intensity of the physical activity, time duration of the physical activity, recommended nutrition, and the like. To that end, the desired similarity between the components of the transient care plan and the components of the initial care plan may be previously determined as a predefined goal of the digital assistant 120.

As an example, the care plan (i.e., the initial care plan) may indicate that the user should perform 25-minute sessions of physical activity four times a week. According to the same example, the user dataset that has been collected with respect to the user may indicate that it would be too intense for the user to perform such physical activity based on the user's current physical fitness. According to the same example, the dataset may further indicate that the preferred physical activity of the user would be practicing yoga or walking in the park. According to the same example, the predefined health-related guidelines of the digital assistant 120 may indicate that outdoor activity shall be performed only when the temperature is within a predetermined range, that the user should drink 6-8 glasses of water per day, and so on. According to the same example, by analyzing the abovementioned example information, transient care plans that would suggest the user to go out for a walk twice a week and practice yoga once a week, may be determined and generated. In the same example, a user response to the transient care plan to ignore suggestion to walk twice a week may be captured by the I/O device 170. To this end, the captured data may be analyzed to further modify the transient care plan to suggest the user to go out for a walk once a week and practice yoga once a week.

In an embodiment, the controller 200, when executing the digital assistant 120, generates the transient care plan based on the result of the analysis. Transient care plans, in the embodiment, are plans that are uniquely determined for a specific user and change over time, either by modifying the existing plans to generate new versions and/or by creating entirely new transient care plans. The transient care plan may include multiple subplans that can collectively makeup the transient care plan. It should be noted that the transient care plan may refer to multiple parameters, such as, the user's mental health, physical health, chronic diseases, social activity, social relationships, and so on.

In an embodiment, the transient care plan may be changed based on a status change of the user, for example, when the user receives new instructions from the medical team, when the medical condition of the user changes, combinations thereof, and the like. That is, the transient care plan may be affected by data inputs that may be received or collected by the I/O device 170 executing the digital assistant 120. Such data inputs may be received/collected using the sensors 140 (that may indicate the user's current state), using the electronic medical record (EMR) of the user (e.g., for collecting updates with respect to the user's health condition), using one or more on-line data sources (e.g., the user's electronic calendar), and the like, and any combination thereof. In an embodiment, the modified transient care plan may include changes in the frequency of executing the transient care plan for projecting the user with certain user activities.

For that matter, the controller 200 may be utilized to constantly or periodically receive/collect data inputs from one or more sources that may change the status of the user with respect to the determined transient care plan. Analysis of data inputs with respect to the dataset of the user and the set of predefined health-related guidelines, and status of the user may be utilized to modify the executed transient care plan. For example, by constantly monitoring the electronic medical record (EMR) of the user, over the network 110, the controller 200 may extract information indicating that the user was discharged from hospital a few minutes ago and therefore, using the TCP engine 280, the controller 200 may be configured to update and modify the transient care plan of the user in accordance with the user's current health condition.

In an embodiment, the controller 200, when executing the digital assistant 120, is configured to generate the transient care plan based on the result of the analysis. It should be noted that the transient care plan may be associated with various aspects of the user's well-being, such as, the user's mental health, physical health, chronic diseases, social activity, social relationships, and so on. In an embodiment, the transient care plan may be changed after a certain duration of time when, for example, the user gets new instructions from the medical team, the medical condition of the user changes, and so on.

According to a further embodiment, the controller 200, when executing the digital assistant 120, may be configured to execute a transient care plan to the user. Executing (or projecting) the transient care plan to the user may be achieved using one or more resources (e.g., the resources 150) that are communicatively connected to and controlled by the digital assistant (e.g., the digital assistant 120). In an embodiment, the controller 200 may be configured to present a transient care plan to the user as, for example, but not limited, to a summary of the transient care plan.

In a further embodiment, the controller 200 may be configured to collect a feedback data from the user with respect to the presented transient care plan. The feedback data may include real-time data that may be collected using one or more sensors (e.g., the sensors 140). The feedback data refers to the user reaction to the presented transient care plan. For example, the user may reject or accept the presented transient care plan or a portion thereof. In an embodiment, the feedback data may be user performance data, which indicates whether the user actually performs the suggested medical instruction projected to the user at a second frequency.

In an embodiment, the controller 200 is also configured to adjust and modify the at least one transient care plan based on the collected feedback data. Adjusting the transient care plan may include for example, changing a recommendation to run three times a week to swim three times a week, since the collected feedback data indicates that swimming is the user's favorite sport. It should be noted that the influence of the feedback data may cause a short-term adjustment of the transient care plan, as discussed above. However, the influence of the feedback data may also cause a long-term adjustment of more general definitions of the digital assistant 120. As an example, for a long-term adjustment: based on the user feedback the controller 200 may determine that in general, no suggestions should be presented to the user before 10 am. According to further embodiment, when adjustment of the transient care plan is desired, the controller 200 may perform an automatic adjustment of the transient care plan without communicating to the user.

According to one embodiment, the controller 200, when executing the digital assistant 120, may be configured to generate a question in order to ask the user, and collect feedback data, regarding one or more preferred care plan components for the transient care plan. For example, an initial care plan may include components relating to physical activity that the user should perform during the week. According to the same example, the value of a 30-minute running component may be equivalent to a 30-minute swimming component and therefore, the controller 200 may be configured to generate a question in order to ask the user what would be the user's choice.

According to one embodiment, the controller 200 may be configured to constantly collect real-time data using the one or more sensors (e.g., the sensors 140). In an embodiment, the controller 200 analyzes the real-time data. The analysis may be achieved using at least one algorithm, such as a machine learning algorithm, that may be stored in a memory (e.g., the memory 220). The algorithm may facilitate determination of at least a current state of at least the user based on the collected real-time data. In a further embodiment, the algorithm may facilitate determination of a current state of the environment near the user (e.g., in a predetermined proximity to the user) based on the real-time data. The current state may reflect the condition of the user and the condition of the environment near the user in real-time, or near real-time. The current state may indicate whether, for example, the user is sleeping, reading, stressed, angry, has company or not, and so on. The current state may further indicate the current time, weather, number of people in the room, people identity, and the like.

In an embodiment, the controller 200 causes the execution of the transient care plan based on, for example, a schedule. A transient care plan may be a reminder to take a certain medication, a suggestion to go out for a 20-minute walk, a suggestion to start practicing yoga, a reminder to drink water, and so on. The transient care plan may include multiple plans. The plans may be executed using one or more resources (e.g., the resources 150) that are communicatively connected to and controlled by the digital assistant (e.g., the digital assistant 120). That is, upon generation of a transient care plan and a schedule that is used for executing at least a portion of the transient care plan, the controller 200 may be configured to execute one or more first plans based on the schedule. For example, the schedule indicates that a suggestion to practicing yoga should be presented to the user at 5 μm, and so when the time is 5 μm the controller 200 executes the plan that includes a suggestion for the user to start practicing yoga.

According to further embodiment, the controller 200 executes a transient care plan, based on at least the current state of the user. Each plan is designed to encourage the user to perform at least a portion of the transient care plan. A plan may be a reminder to take a certain medication, a suggestion to go out for a 20-minute walk, a suggestion to start practicing yoga, a reminder to drink water, and so on. A plan may also include a specific manner, approach, tone, and the like, to be used for presenting a certain reminder, suggestion, and the like, to the user. To that end, the controller 200 may analyze the dataset of the user and the real-time data that is indicative of the current state in order to determine the most appropriate plan, approach, tone, and the like, with respect to the specific user and the specific scenario. Such determination may be achieved by applying one or more machine learning algorithms to the dataset of the user and to the real-time data. It should be noted that determining or selecting the most appropriate approach, such as a tone, may be useful for encouraging the user to perform and fulfill one or more required user activities (e.g., playing a cognitive game, call a friend, go out for a walk). As a non-limiting example, although a predetermined schedule determines that a reminder to take a certain medication should be presented to the user within one minute, the current state indicates that the user has company at home so the execution of the reminder may be postponed.

According to another embodiment, the controller 200 may be configured to collect a feedback data from the user with respect to the executed transient care plan. That is, after the plan is executed already, a feedback data may be collected. According to further embodiment, the transient care plan may be adjusted by the controller 200 based on the feedback data that is indicative of the user's reaction to the executed at least one plan.

In an embodiment, the controller 200 may be configured to determine whether there is a first gap between the initial care plan of the user and the transient care plan. The first gap is a difference in at least one aspect between the care plan (i.e., the initial care plan) and the transient care plan. The first gap may indicate that the instructions that were received from the medical provider (e.g., doctor), and the transient care plans that were determined by the digital assistant 120 are not synchronized. In an embodiment, the gap between initial care plan and the transient care plan may be determined based on the first frequency and the second frequency of the initial care plan and the transient care plan, respectively. In an embodiment, the controller 200 may be configured to indicate the specific differences between the initial care plan and the transient care plan. As an example, while the care plan indicates that the user is required to perform physical activity three times a week, the generated transient care plan may include asking the user to perform physical activity only twice a week. In one embodiment, the transient care plan may be adjusted based on the determined first gap.

In another embodiment, the controller 200 may be configured to generate a first electronic report based on the determined first gap between the initial care plan and the transient care plan. The first electronic report may include a description of the first gap and the reasons for the first gap. Then, the first electronic report may be sent over a network (e.g., the network 110) to a computing device (e.g., the computing device 180) that is associated with the medical provider. By sending the first electronic report to the medical provider, the medical provider may receive information about the health-related instructions the digital assistant 120 generated for the user to follow and perform.

In an embodiment, the controller 200 may be configured to determine a second gap between the transient care plan and a user performance with respect to the executed transient care plan by the I/O device 170 executing the digital assistant 120. In an embodiment, the user performance data indicates the user's actual response to the executed transient care plan that projects the instructions based on the second frequency. The second gap is a difference in at least one aspect between the transient care plan and the user performance with respect to the execution of the transient care plan. Such determination may be achieved by collecting and analyzing real-time data indicating that the user performed according to the projected transient care plan (e.g., suggestion) or not, and comparing the analyzed real-time data to the transient care plan. Thus, in case there is a gap between the planned transient care plan and the plans that were eventually accepted and performed by the user, such a gap (i.e., the second gap) may be detected.

According to one embodiment, the transient care plan may be adjusted based on the second gap. According to another embodiment, the controller 200 may be configured to generate a second electronic report based on the determined second gap between the transient care plan and the user performance in response to the executed transient care plan. The second electronic report may include a description of the second gap and the reasons for the second gap. Then, the second electronic report may be sent over a network (e.g., the network 110) to a computing device (e.g., the computing device 180) that is associated with the medical entity. By sending the second electronic report to the medical provider, the medical provider receives information about the plans that were performed by the user compared to the determined transient care plans.

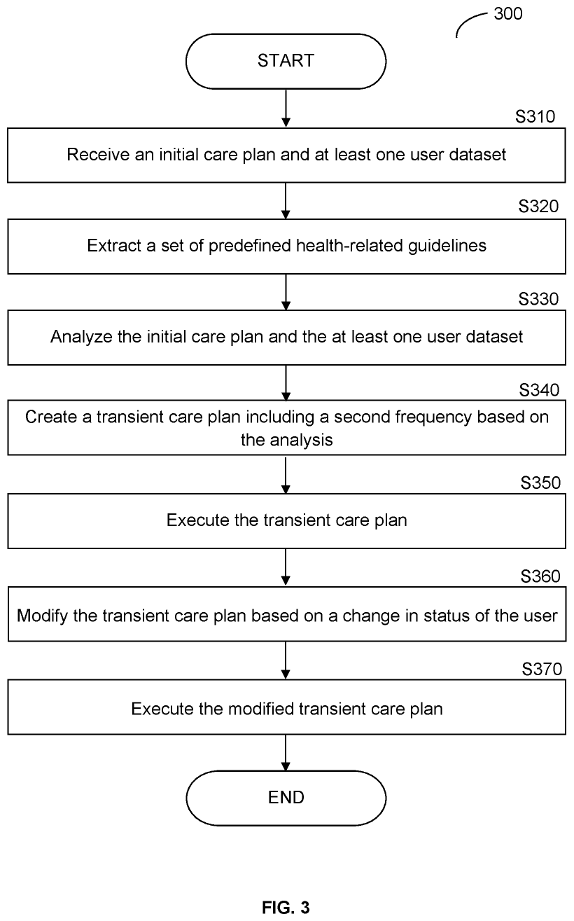

FIG. 3 shows an example flowchart 300 of a method for executing and modifying a transient care plan for a user of the digital assistant according to an embodiment. The method described herein may be executed by the controller 200 that is further described herein above with respect to FIG. 2.

At S310, an initial care plan and at least one user dataset related to a user are collected. The initial care plan is received from a computing device that is associated with a medical provider (e.g., a doctor, medical team). The initial care plan may include medical instructions such as, take a certain medication at a specific timing, do physical activity three times a week, avoid red meat, and so on, to be executed a certain number of times as defined by a first frequency. The dataset of the user of the digital assistant may include at least historical data of the user (such as, preferences, behavioral patterns, etc.). In an embodiment, the dataset may also include real-time data that is being collected in real-time with respect to the user and the user's environment. The dataset of the user may be collected using sensors (e.g., the sensors 140) that are communicatively connected to and controlled by the digital assistant 120.

At S320, a set of predefined health-related guidelines of the digital assistant 120 may be extracted. The set of predefined health-related guidelines of the digital assistant 120 may refer to predetermined rules. Such health-related guidelines may indicate, for example that, the user should drink 6-8 glasses of water per day, the user should sleep at least 7 hours at night, and so on.

At S330, the initial care plan and the at least one user dataset are analyzed. The initial care plan is analyzed with respect to the at least one dataset of the user and the set of predefined health-related guidelines. The analysis of the care plan with the dataset of the user and the set of predefined health-related guidelines may be achieved by applying at least one algorithm, such as a machine learning algorithm, that is adapted to determine a transient care plan.

At S340, a transient care plan is created based on the analysis. In an embodiment, the transient care plan may include a plurality of instructions for a user activity to be executed at a second frequency. In an embodiment, the transient care plan may include a plurality of regimens (e.g., suggestions, reminders, and the more) that may be applicable for the respective user determined to be executed via an I/O device (e.g., the I/O device 170, FIG. 1) at the second frequency. In an embodiment, the transient care plan may be associated with various aspects of the user's well-being such as, the user's mental health, physical health, chronic diseases, social activity, social relationships, and so on.

At S350, the created transient care plan is executed. The transient care plan is executed by projecting the plurality of instructions to perform the user activity based on the second frequency. As a non-limiting example, when the second frequency is twice a week, an instruction to perform the user activity is projected from the I/O device two times within a 7 day period. The transient care plan may be designed to encourage the user to perform at least one of the plurality of instructions of the transient care plan at certain frequencies. In an embodiment, the transient care plan may be projected to a user in a certain manner, approach, tone, time of day, day of the week, and the like, and any combination thereof, that is customized to the specific user. Such customization may be based on the user's, for example, but is not limited to, preferences, historical information, behavioral patterns, and the like, and any combination thereof.

At S360, the transient care plan is modified based on a change in status of the user. The change in status of the user may be determined by analyzing user datasets collected via the I/O device (e.g., the I/O device 170, FIG. 1) with respect to the executed transient care plan. The analysis may be achieved using one or more computer vision techniques, audio signal processing techniques, machine learning techniques, and the like.

In an embodiment, the transient care plan is modified according to the determined change in status of the user to execute the transient care plan based on a third frequency. In further embodiment, modifying the transient care plan may include, for example, changing a recommendation, remove a certain reminder or recommendation, changing the intensity and duration of time for a certain reminder, and the like, and any combination thereof.

In an embodiment, the user datasets may include, but are not limited to, updates from the electronic medical record (EMR), data from the medical provider, and the like. In a further embodiment, the user dataset may include feedback data indicating user reaction to the executed transient care plan. For example, the user may reject or accept the executed plan, or may fail to respond to the plan. In a further embodiment, the user dataset may include real-time data that may be collected using one or more sensors (e.g., the sensors 140, FIG. 1).

At S370, the modified transient care plan is executed. The modified transient care plan is executed based on the third frequency that was updated according to the change in user status. As a non-limiting example, when the change in user status indicates that the user is scheduled for surgery and should lose weight, the transient care plan may be modified to increase a frequency of walking from twice a week to five times a week and the instructions to walk may be projected five times within a seven day period. In an embodiment, the execution of the transient care plan may be performed by projecting to a use via the I/O device (e.g., the I/O device 170, FIG. 1).

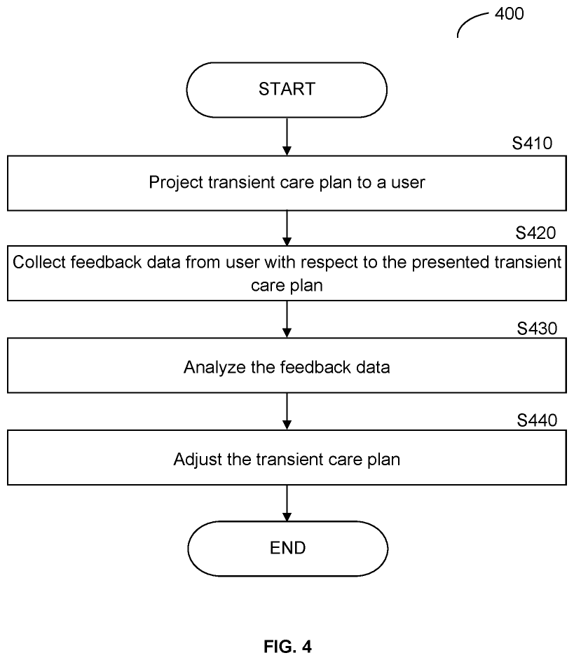

FIG. 4 shows an example flowchart 400 of a method for adjusting a transient care plan based on feedback data received from the user of the digital assistant with respect to the presented transient care plan according to an embodiment. The method described herein may be executed by the controller 200 that is further described herein above with respect to FIG. 2.

At S410, a transient care plan that was previously generated (further described in FIGS. 2 and 3) is presented to the user. The transient care plan may be presented to the user by one or more resources (e.g., the resources 150, FIG. 1). As further discussed herein above, the transient care plan may refer to as a health regimen that is customized to the user based on, among other things, the user's behavior, patterns, habits, preferences, and the like. The transient care plan includes the plurality of instructions and the second frequency at which the instructions should be presented to the user. Presenting the transient care plan may include, but is not limited to, displaying or otherwise projecting a summary of the transient care plan including information such as the user activities to be performed, the frequency at which each activity is to be performed, and the like.

At S420, feedback data is collected from the user with respect to the presented transient care plan. The feedback data refers to the reaction of the user with respect to the presented transient care plan. For example, the user may reject or accept the transient care plan. In an embodiment, the feedback data may include real-time data that may be collected using one or more sensors (e.g., the sensors 140, FIG. 1) and include, without limitation, images, videos, audio signals, and the like.

At S430, the collected feedback data is analyzed. The analysis of the feedback data may be achieved by applying one or more computer vision techniques, audio signal processing techniques, machine learning techniques, and the like.

At S440, the transient care plan is adjusted based on the collected feedback data. In an example embodiment, adjusting the transient care plan may include, for example, changing a recommendation provided by the medical provider to run three times a week to run only twice a week for six weeks, and then run three times a week as originally determined by the initial care plan.

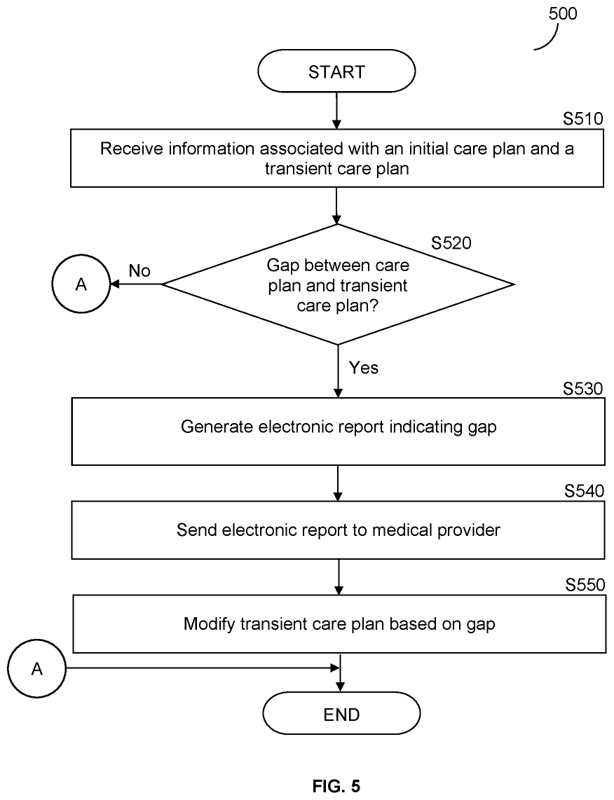

FIG. 5 shows an example flowchart 500 of a method for detecting a first gap between an initial care plan and a transient care plan and for generating a corresponding electronic report according to an embodiment. The method described herein may be executed by the controller 200 that is further described herein above with respect to FIG. 2.

At S510, information associated with a care plan (i.e., an initial care plan) and information that is associated with a transient care plan that was generated for the user, is received. It should be noted that the initial care plan may be received from, for example, a computing device 180 (e.g., the computing device 180, FIG. 1) associated with a medical provider, and the transient care plan may be previously created for the user based on the analysis that is further discussed herein above with respect to FIG. 2.

At S520, it is checked whether a first gap between the initial care plan and the transient care plan is detected and if so, execution continues with S530; otherwise, execution terminates. The first gap may indicate that the medical instructions received from the medical provider (e.g., the doctor) and the instructions that were generated by the digital assistant 120 as a transient care plan are not synchronized. In an embodiment, the first gap may be determined by comparing a first frequency of the initial care plan with a second frequency of the transient care plan. Each frequency indicates how many times the user activity associated with medical instruction should be performed by the user, and thus, be projected to a user via an I/O device (e.g., the I/O device 170, FIG. 1).

In an embodiment, the controller 200 may be configured to indicate the specific difference between the initial care plan and the transient care plan, e.g., by comparing components of the care plan to the components of the transient care plan. For example, while the care plan indicates that the user is required to perform physical activity three times a week, the transient care plan may include asking the user to perform physical activity only twice a week.

At S530, a first electronic report may be generated based on the determined first gap. The first electronic report may include a description of the first gap between the initial are plan and the transient care plan and the reasons for the first gap.

At S540, the first electronic report is sent to a predefined computing device (e.g., the computing device 170, FIG. 1), for example, of a medical provider. As noted above, the first electronic report may include a description of the first gap and the reasons for the first gap.

At optional S550, the transient care plan may be modified based on the first gap. The modification may be performed by the controller 200, executing the digital assistant 120, using the transient care plan (TCP) engine 280. The transient care plan may be modified to include more components that are similar, or identical, to the components of the original care plan. In an embodiment, the transient care plan may be modified in order to synchronize the transient care plan with the initial care plan. As a non-limiting example, when the gap is a difference in frequency between an initial care plan having a first frequency of twice per week and a transient care plan having a second frequency of once per week, the transient care plan may be modified to have a second frequency of twice per week.

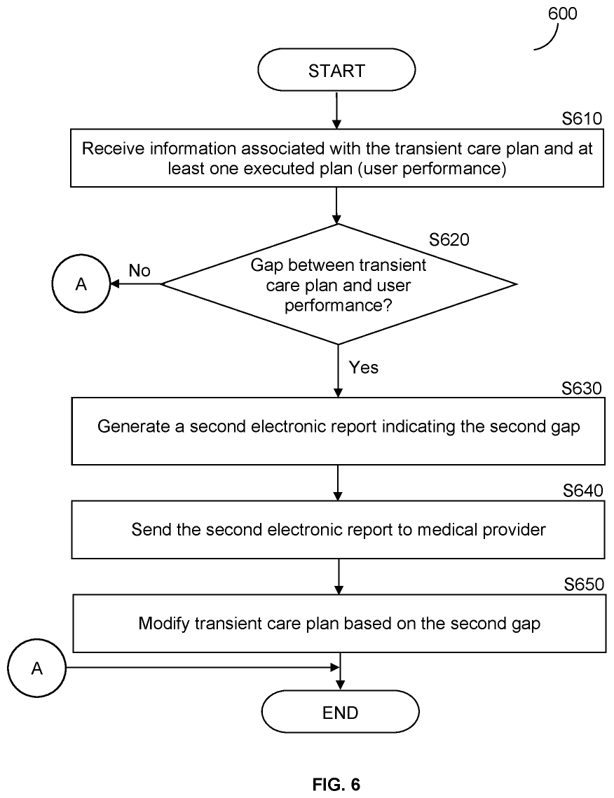

FIG. 6 shows an example flowchart 600 of a method for detecting a second gap between a transient care plan and a user performance data in response to the transient care plan that was executed by an I/O device according to an embodiment. The method described herein may be executed by the controller 200 that is further described herein above with respect to FIG. 2.

At S610, information associated with a transient care plan that was generated for a user and user performance data with respect to a transient care plan that was executed based on a second frequency are received. It should be noted that the transient care plan may be previously generated for the user based on the analysis that is further discussed herein above with respect to FIG. 2. As noted herein, a transient care plan may be designed to encourage the user to perform a user activity based on at least one of the plurality of instructions and a second frequency. The user performance data indicates actual user actions in response to the projected transient care plan. That is, the user performance data may indicate whether the user followed the instructions of the transient care plan or not, to what extent the user followed the instructions (e.g., how many times within a given time period as would be expected based on the second frequency for that time period). In an embodiment, the user performance data is collected via an I/O device (e.g., the I/O device 170, FIG. 1). In a further embodiment, the user performance data may include real-time data that may be collected using one or more sensors (e.g., the sensors 140, FIG. 1) and include, without limitation, images, videos, audio signals, and the like.

At S620, it is checked whether a second gap is detected between the transient care plan and user performance data, and if so, execution continues with S630; otherwise, execution terminates. The second gap may indicate that the transient care plan and the user performance data, indicating what was actually performed by the user, are not synchronized. In an embodiment, the controller 200 may be configured to indicate the specific differences between the transient care plan and the user performance data. In a further embodiment, the specific differences may include, but are not limited to, differences in frequency, in user activities to be performed, in intensity, in duration of time, combinations thereof, and the like. For example, while the transient care plan indicates that the user is required to perform physical activity five times a week, the user had only performed the physical activity twice over the last week according to collected user performance data such that there is a gap in frequency between the transient care plan and the user performance.

At S630, a second electronic report may be generated based on the determined second gap of the transient care plan and the user performance data. The second electronic report may include a description of the second gap and the reasons for the second gap.

At S640, the second electronic report is sent to a predefined computing device (e.g., the computing device 180, FIG. 1). As noted above, the second electronic report may include a description of the second gap and the reasons for the second gap.

At optional S650, the transient care plan may be modified. The modification may be performed by the controller 200 using the transient care plan (TCP) engine 280. The transient care plan may be modified, for example, based on the actual user performance data in response to the executed transient care plan via the I/O device (e.g., the I/O device 170, FIG. 1). In a further embodiment, the transient care plan may be modified based on, the user performance data, the executed transient care plan, the initial care plan, a combination thereof, and the like.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

What is claimed is:

1. A method for executing transient care plans for a user via an input/output (I/O) device, comprising:
analyzing an initial care plan of a user and at least one user dataset captured by an input/output (I/O) device, wherein the initial care plan of the user is a health regimen uniquely determined for the user indicating at least one medical instruction defined at least with respect to a first frequency of a user activity;
creating a transient care plan to be executed via the I/O device based on the analysis, wherein the transient care plan defines a second frequency at which a plurality of instructions to perform the user activity is to be projected via the I/O device, wherein the second frequency is determined based on the first frequency and the at least one user dataset captured by the I/O device;
executing the transient care plan by projecting the plurality of instructions to perform the user activity via the I/O device based on the second frequency;
modifying the transient care plan based on data indicating a change in status of the user, wherein the modified transient care plan defines a third frequency at which a plurality of instructions to perform the user activity is to be projected via the I/O device; and
executing the modified transient care plan by projecting the plurality of instructions to perform the user activity via the I/O device based on the third frequency.

2. The method of claim 1, wherein the transient care plan is modified based further on at least one response of the user to the execution of the transient care plan, wherein the at least one response of the user is captured by the I/O device.

3. The method of claim 2, wherein the at least one response of the user includes real-time user data and real-time environment data captured by at least one sensor of the I/O device.

4. The method of claim 1, further comprising:
applying a machine learning model trained to determine a current state of the user based on real-time user data and real-time environment data for an environment within a predetermined proximity of the user, wherein the real-time user data and the real-time environment data are captured by at least one sensor of the I/O device, wherein the transient care plan is modified based further on the determined current state.

5. The method of claim 1, further comprising:
determining, based on the first frequency and the second frequency, a gap between the initial care plan of the user and the transient care plan, wherein the gap between the initial care plan and the transient care plan includes at least one difference between the initial care plan and the transient care plan, wherein the transient care plan is modified based further on the determined gap.

6. The method of claim 5, wherein the transient care plan is modified based on the determined gap between the initial care plan and the transient care plan in order to synchronize the transient care plan with the initial care plan.

7. The method of claim 5, further comprising:
generating an electronic report indicating the gap between the initial care plan and the transient care plan, wherein the electronic report includes the at least one difference between the care plan and the transient care plan, wherein each of the at least one difference is a difference with respect to at least one of: a frequency, an activity, an intensity, and a duration of time; and
providing the electronic report to a medical provider user device.

8. The method of claim 1, further comprising
collecting user performance data indicating a user performance with respect to the execution of the transient care plan projected by the I/O device, wherein the user performance indicates whether the user adhered to the plurality of instructions of the transient care plan executed based on the second frequency; and
determining a gap between the transient care plan and the user performance data, wherein the gap between the transient care plan and the user performance data indicates at least one difference between the transient care plan and the user performance data, wherein the transient care plan is modified based further on the gap between the transient care plan and the user performance data.

9. The method of claim 8, further comprising:
generating an electronic report based on the gap between the transient care plan and the user performance data, wherein the electronic report includes the at least one difference between the transient care plan and the user performance data, wherein each of the at least one difference is a difference with respect to at least one of: a frequency, an activity, an intensity, and a duration of time; and
providing the electronic report to a medical provider user device.

10. A non-transitory computer readable medium having stored thereon instructions for causing a processing circuitry to execute a process, the process comprising:
analyzing an initial care plan of a user and at least one user dataset captured by an input/output (I/O) device, wherein the initial care plan of the user is a health regimen uniquely determined for the user indicating at least one medical instruction defined at least with respect to a first frequency of a user activity;
creating a transient care plan to be executed via the I/O device based on the analysis, wherein the transient care plan defines a second frequency at which a plurality of instructions to perform the user activity is to be projected via the I/O device, wherein the second frequency is determined based on the first frequency and the at least one user dataset captured by the I/O device;
executing the transient care plan by projecting the plurality of instructions to perform the user activity via the I/O device based on the second frequency;
modifying the transient care plan based on data indicating a change in status of the user, wherein the modified transient care plan defines a third frequency at which a plurality of instructions to perform the user activity is to be projected via the I/O device; and
executing the modified transient care plan by projecting the plurality of instructions to perform the user activity via the I/O device based on the third frequency.

11. A system for executing transient care plans for a user via an input/output (I/O) device, comprising:
a processing circuitry; and
a memory, the memory containing instructions that, when executed by the processing circuitry, configure the system to:
analyze an initial care plan of a user and at least one user dataset captured by an input/output (I/O) device, wherein the initial care plan of the user is a health regimen uniquely determined for the user indicating at least one medical instruction defined at least with respect to a first frequency of a user activity;
create a transient care plan to be executed via the I/O device based on the analysis, wherein the transient care plan defines a second frequency at which a plurality of instructions to perform the user activity is to be projected via the I/O device, wherein the second frequency is determined based on the first frequency and the at least one user dataset captured by the I/O device;
execute the transient care plan by projecting the plurality of instructions to perform the user activity via the I/O device based on the second frequency;
modify the transient care plan based on data indicating a change in status of the user, wherein the modified transient care plan defines a third frequency at which a plurality of instructions to perform the user activity is to be projected via the I/O device; and
execute the modified transient care plan by projecting the plurality of instructions to perform the user activity via the I/O device based on the third frequency.

12. The system of claim 11, wherein the transient care plan is modified based further on at least one response of the user to the execution of the transient care plan, wherein the at least one response of the user is captured by the I/O device.

13. The system of claim 12, wherein the at least one response of the user includes real-time user data and real-time environment data captured by at least one sensor of the I/O device.

14. The system of claim 11, wherein the system is further configured to:
apply a machine learning model trained to determine a current state of the user based on real-time user data and real-time environment data for an environment within a predetermined proximity of the user, wherein the real-time user data and the real-time environment data are captured by at least one sensor of the I/O device, wherein the transient care plan is modified based further on the determined current state.

15. The system of claim 11, wherein the system is further configured to:
determine, based on the first frequency and the second frequency, a gap between the initial care plan of the user and the transient care plan, wherein the gap between the initial care plan and the transient care plan includes at least one difference between the initial care plan and the transient care plan, wherein the transient care plan is modified based further on the determined gap.

16. The system of claim 15, wherein the transient care plan is modified based on the determined gap between the initial care plan and the transient care plan in order to synchronize the transient care plan with the initial care plan.

17. The system of claim 15, wherein the system is further configured to:
generate an electronic report indicating the gap between the initial care plan and the transient care plan, wherein the electronic report includes the at least one difference between the care plan and the transient care plan, wherein each of the at least one difference is a difference with respect to at least one of: a frequency, an activity, an intensity, and a duration of time; and
provide the electronic report to a medical provider user device.

18. The system of claim 11, wherein the system is further configured to
collect user performance data indicating a user performance with respect to the execution of the transient care plan projected by the I/O device, wherein the user performance indicates whether the user adhered to the plurality of instructions of the transient care plan executed based on the second frequency; and
determine a gap between the transient care plan and the user performance data, wherein the gap between the transient care plan and the user performance data indicates at least one difference between the transient care plan and the user performance data, wherein the transient care plan is modified based further on the gap between the transient care plan and the user performance data.

19. The system of claim 18, wherein the system is further configured to:
generate an electronic report based on the gap between the transient care plan and the user performance data, wherein the electronic report includes the at least one difference between the transient care plan and the user performance data, wherein each of the at least one difference is a difference with respect to at least one of: a frequency, an activity, an intensity, and a duration of time; and
provide the electronic report to a medical provider user device.
